(19) United States
(12) Patent Application Publication
(10) Pub. No.: US 2009/0197956 A1
(43) Pub. Date: Aug. 6, 2009

(54) TREATMENT OF ACUTE OTITIS MEDIA WITH XYLITOL AND N-ACETYLCYSTEINE

(75) Inventors: Jarrett T. Disbrow, Raleigh, NC (US); Joshua R. Disbrow, Wake Forest, NC (US)

Correspondence Address: PASSE' INTELLECTUAL PROPERTY, LLC, 1717 BRASSFIELD RD., RALEIGH, NC, 27614, US

(73) Assignee: ARBOR PHARMACEUTICALS, INC., Raleigh, NC (US)

(21) Appl. No.: 12/025,563

(22) Filed: Feb. 4, 2008

(51) Int. Cl. A61K 31/195; A61P 27/16

(52) U.S. Cl. 514/561

(57) ABSTRACT

The present invention relates to the discovery that the treatment of Acute Otis Media (AOM) can be accomplished by a synergistic combination of xylitol and N-acetylcysteine. The treatment with this combination shows that symptoms of AOM can be decreased and or treated to a greater degree than know with xylitol alone. The present invention includes both pharmaceutical compositions and methods of treatment.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to the treatment of individuals diagnosed with acute otitis media (AOM). In particular the present invention relates to the treatment of AOM with a synergistic combination of xylitol and N-acetylcysteine.

2. Description of Related Art

Otitis media is an inflammation of the middle ear, the space behind the ear drum. It is one of the two conditions that are commonly thought of as ear infections, the other being Otitis externa. The inflammation of the middle ear in otitis media is caused by a bacterial or viral infection. Ear infections are very common in childhood, and include acute and recurrent—or chronic-conditions; all of which involve inflammation of the ear drum (tympanic membrane), and are usually associated with a buildup of fluid in the space behind the ear drum (middle ear space). This inflammation and fluid buildup results in pain which causes caregivers or patients to readily seek medical attention for the condition.

Inflammation of the skin of the ear canal, and the associated pain, are the essence of all Otitis infections. If inflammation progresses to infection, the ear canal may fill with swollen tissue and drainage. Once the ear canal is blocked, hearing will be dampened (conductive hearing impairment) until the condition improves. In many individuals, for the reasons discussed below, the condition is recurrent and will happen several times in a lifetime (chronic or recurrent Otitis media).

There are essentially two types of otitis media recognized, each with a separate diagnosis and separate causes and general symptomatic profiles. Otitis Media with Effusion (OME) is primarily a non specific inflammatory response characterized by fluid behind the ear. Acute Otis Media (AOM) without effusion is an infectious disease characterized by rapid onset, pain, inflammation and the like of the middle ear. It appears that 40 to 50% of AOM in young children are caused by Streptococcus pneumonia, 20 to 30% by Haemophilus influenza and 10 to 15% by Moraxella catarrhalis. Recurrent AOM has been associated with excessive levels of S. pneumoniaas a result of some environmental factors. Additionally, low levels of antioxidants, including glutathione in particular, have also been shown to correlate with recurrent AOM-Oxidant and antioxidant levels in children with acute otitis media and tonsillitis: A comparative study. International Journal of Pediatric Otorhinolaryngology, Volume 69, Issue 6, Pages 823-827 M. Cemek, S. Dede, F. Bayiro\check{g}lu, H. Caksen, F. Cemek, K. Yuca.

Treatment of AOM has classically been accomplished with either antibacterial medications, steroids to treat inflammation or both. Classic antibacterial treatment based on the particular organism and its susceptibility to the antibiotic rather than the disease state has been the mainstay of most bacterial type infections. In U.S. Pat. No. 6,987,093 to Dunne (and assigned to Pfizer) issued Jan. 17, 2006 discloses the use of Azithromycin to treat AOM. Of course a problem with antibiotic use is the eventual resistance of the particular antibiotic to the strains that cause AOM. It is clear that many high dosage antimicrobial treatments have a number of untoward side effects.

One non-antibacterial approach that has been introduced is the use of Xylitol. Xylitol, a natural sweetener, was first used medicinally around World War II after its discovery in Finland of improved health of subjects using it. Chewing gums are almost universally sweetened with xylitol in Finland and most of Europe, since in addition to its known anti bacterial activity it has anti-carie activity. In spite of the fact that xylitol containing gums have been used for several decades, U.S. Pat. Nos. 6,066,677 and 5,719,196 to Uhari were issued to the prevention and treatment of AOM by administration of xylitol by chewing xylitol containing gum. N-acetylecysteine (NAC) is used mainly for its mucolytic activity, and other properties including expectorant activity, bronchorrheic activity, mucoregulatory action, ability to regulate reduced glutathione levels, ability to stimulate immune systems debilitated by HIV virus and its activity as a free radical scavenger.

In U.S. Pat. No. 5,080,906 to Carenzi there is disclosed a composition for more effectively delivering orally administered NAC by addition of tris (hydroxymethyl)aminomethane. While sweeteners are included in the described formula xylitol is neither shown by example nor suggested. Also, use of NAC for the treatment of AOM (either acutely or recurrent disease) is not taught nor suggested While administration of oral and inhaled NAC has been studied for its effects in otitis media with effusion, its use is not taught or suggested in acute otitis media—a distinct condition with unique pathophysiology, symptoms, and sequelea.

While the above information and treatments are useful, it would be even more useful if more effective and less tolerance resistant formulations were available for the treatment of AOM.

### SUMMARY OF THE INVENTION

It has been discovered that there is a synergistic effect when Xylitol is combined with NAC and used to treat or prevent occurrences of AOM. NAC has not previously been used to treat AOM and the combination of the two drugs yields results that are superior to the treatment of using Xylitol alone. The results are that either there is a higher degree of preventative effect or that the symptoms of AOM can be decreased or treated to a greater degree than with Xylitol alone. Prior to the present invention NAC has not been shown to have value in the treatment of AOM.

Accordingly, in one embodiment the present invention provides a pharmaceutical composition comprising an admixture of xylitol and N-acetylcysteine formulated with amounts and in a ratios effective for the treatment of Acute Otitis Media by oral administration.

In yet another embodiment of the present invention there is a method of treating Acute Otitis Media in a human comprising orally administering to the human an effective amount of a combination xylitol and N-acetylcysteine or its pharmaceutically acceptable salts. Specific pharmaceutically acceptable N-acetylcysteine salts including but not limited to the lysine salt of N-isobutyryl-L-cysteine, the ethylenediamine salt of N-isobutyryl-L-cysteine, and the diethanolamine salt of N-isobutyryl-L-cysteine may be utilized in combination with xylitol to enable improved composition and bioavailability of the composition Xylitol is administered in daily doses of 1 g up to 20 g daily and combined with N-acetylcysteine in administered daily doses of 10 mg up to 2 g The combination will be commonly administered in a range of ratios of 8.4-10 g xylitol for every 200-600 mg N-acetylcysteine.

These and other objects of the present invention will be clear when taken in view of the detailed specification and disclosure.

### DETAILED DESCRIPTION OF THE INVENTION

The general description of the invention and how to use the present invention is stated in the Brief Summary above. This detailed description defines the meaning of the terms used herein and specifically describes embodiments in order for those skilled in the art to practice the invention. The above interests in successfully treating ear infections can be seen from the disclosure which follows and are met by the present invention.

While this invention is susceptible of embodiment in many different forms, there will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

The terms “a” or “an”, as used herein, are defined as one as or more than one. The term “plurality”, as used herein, is defined as two or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, “and an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means “any of the following: A; B; C; A and B; A and C; B and C; A, B and C”. An exception to this definition will occur only when a combination of elements, functions, steps or acts are in some way inherently mutually exclusive.

As used herein “treatment” or “treating” refers to at least an amelioration (or reduction) and/or the dietary management of and/or preventing the symptoms associated with Acute Otis Media including those of chronic—or recurrent-versions of AOM. Amelioration and/or dietary management and/or prevention are used in a broad sense to refer to at least a reduction in the magnitude of a parameter, e.g. symptom, such as level or magnitude of fever, inflammation, onset, duration, number of episodes, etc. As such, treatment also includes situations where the pathological condition, or at least symptoms associated therewith, are completely or partially inhibited, e.g. prevented from happening, or stopped, e.g. terminated, such that the host no longer suffers from the condition, or at least the symptoms that characterize the condition.

A variety of hosts are treatable according to the methods of the invention. Generally, such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

As used herein “effective” amount refers to a dosage sufficient to show an effective response as described above. The dosage may be changed based on such things as severity, uses as a treatment or preventative, patient age or size and the like but in general effective ranges and rations are within the skill in the art in light of this discloser. In one embodiment the dosage of about 2 to 10 grams per day of xylitol will be effective. It is possible that in some cases the synergistic effect of the present invention may provide an opportunity to provide a dosage between about 2 and less than about 6 grams per day.

An effective dosage of NAC to combine with xylitol will depend in part on the dosage of xylitol selected. In one embodiment the amount of NAC is from about 10 mg to about 2 grams per day. In another embodiment the amount is from about 100 mg to about 1.5 grams per day and in yet another embodiment it is between about 200 mg and 600 mg per day.

The compositions which are the object of the present invention are prepared using methods well known in the art to mix the NAC and xylitol together with other pharmaceutically acceptable flavorings, excipients and other additives as may be desired for administration.

The compositions described above may be combined with a pharmaceutical carrier in accordance with known techniques to provide a pharmaceutical formulation useful for carrying out the methods described above. See, e.g., Remington, The Science and Practice of Pharmacy (9.sup.th Ed. 1995). In the manufacture of a pharmaceutical formulation according to the invention, the active compounds (including their physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a syrup but may also be acceptable in a unit-dose formulation, for example, a tablet, capsule, or chewable form, which may contain from 0.01 or 0.5% to 95% or 99% by weight of the active compound. One or more active compounds may be incorporated in the formulations of the invention, which may be prepared by any of the well known techniques of pharmacy consisting essentially of admixing the components, optionally including one or more accessory ingredients.

Formulations suitable for oral administration may be presented in discrete units, such as capsules, dissolvable films, cachets, lozenges, chewables (including gums and other chewable forms), or the like, each containing a predetermined amount of the active compound; as a powder or granules; as a solution, syrup or a suspension in an aqueous or non-aqueous liquid; or as an oil-in-water or water-in-oil emulsion. For the purposes of this invention inhaled formulations shall be considered an orally administered formulation when claimed as oral administration. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture.

The formulations of compositions of the present invention can be used to treat acute otitis media without ear effusion. Diagnosis of AOM could be accomplished by the cultured presence of AOM bacterial pathogens as described above. Administration could be between 1 and about 5 times a day splitting the daily dose into appropriately smaller doses. In treatment, prevention or dietary management of a disease state a relatively higher dosage might be used while in prophylaxis of AOM a relatively smaller dosage could be employed. The levels of each active ingredient will also be adjusted based on the dosage frequency (e.g. once daily up to five times daily).

### EXAMPLES

### Example 1

### Formulation of a Composition of the Invention

The following example depicts one formulation of the current invention. A suitable daily dose of 10 g xylitol is divided into three doses containing 3.34 g xylitol and is effectively mixed and suspended in a taste-masked syrup containing 133.33 mg of N-acetylcysteine to yield a total daily dosage/volume of pharmaceutically active ingredients of 10 g xylitol and 400 mg N-acetylcysteine. The composition is delivered in a sweet suspension or syrup comprised of pharmaceutically acceptable excipients and/or stabilizing agents, and the product is administered 3 times daily.

### Example 2

### Formulation of a Composition of the Invention

The following example depicts another formulation of the current invention. A suitable daily dose of xylitol is delivered via a single dose containing 10 g xylitol and is effectively mixed and suspended in a taste-masked syrup containing 400 mg of N-acetylcysteine. The composition is delivered in a sweet suspension or syrup comprised of pharmaceutically acceptable excipients and/or stabilizing agents, and the product is administered once daily.

### Example 3

### The Following Example Depicts Another Formulation of the Current Invention.

A suitable daily dose of 10 g xylitol is divided into five doses containing 2 g xylitol and is effectively embedded in a dissolvable film or lozenge containing 80 mg of N-acetylcysteine, again yielding a daily dosage/volume of pharmaceutically active ingredients of 10.0 g xylitol and 400.0 mg of N-acetylcysteine. The dissolvable film yields clinical benefit comparable to syrup or suspension and is consumed orally 5 times daily.

### Example 4

### The Following Example Depicts Another Formulation of the Current Invention.

A suitable daily dose of 10 g xylitol if divided into 3 doses containing 3.34 g xylitol and is effectively mixed and suspended in a taste-masked syrup containing 133.33 mg of N-acetylcysteine to yield a total daily dosage/volume of pharmaceutically active ingredients of 10 g xylitol and 400 mg N-acetylcysteine. The detailed composition is delivered in a congealed “gummie” candy type preparation comprised of pharmaceutically acceptable excipients and/or stabilizing agents, and the product is administered 3 times daily. The composition is consumed orally.

What is claimed is:

1. A pharmaceutical composition comprising an admixture of xylitol and N-acetylcysteine formulated with amounts and in a ratio effective for the treatment of Acute Otitis Media.

2. A composition according to claim 1 wherein the composition is formulated such that it can deliver between about 1 and about 20 grams of xylitol per day when taken between 1 and 5 times per day.

3. A composition according to claim 1 wherein the composition is formulated such that it can deliver between about 10 mg and 2 grams of N-acetylcysteine per day when taken between 1 and 5 (five) times per day.

4. A composition according to claim 1 wherein the composition is a formulation for oral administration.

5. A composition according to claim 4 wherein the composition is orally formulated in a chewable formulation.

6. A composition according to claim 5 selected from the group of chewing gum and chewable candy.

7. A composition according to claim 4 wherein the composition is formulated in a dissolvable film.

8. A method of treating Acute Otitis Media in a human comprising orally administering to the human an effective amount of a combination of xylitol and N-acetylcysteine.
